(19) United States
(12) Patent Application Publication
(10) Pub. No.: US 2024/0335674 A1
(43) Pub. Date: Oct. 10, 2024

(54) PHOTOTHERAPY DEVICE AND CONTROL METHOD THEREFOR

(71) Applicants: Kwang Chul HYUN, Gwangju-si, Gyeonggi-do (KR); INHA UNIVERSITY RESEARCH AND BUSINESS FOUNDATION, Incheon (KR)

(72) Inventors: Kwang Chul HYUN, Gwangju-si (KR); Jun Kyu SUH, Seoul (KR); Ji Kan RYU, Incheon (KR)

(73) Assignees: Kwang Chul HYUN, Gwangju-si, Gyeonggi-do (KR); INHA UNIVERSITY RESEARCH AND BUSINESS FOUNDATION, Incheon (KR)

(21) Appl. No.: 18/692,800

(22) PCT Filed: May 9, 2022
(86) PCT No.: PCT/KR2022/006537

§ 371 Date: Apr. 3, 2024

(30) Foreign Application Priority Data
Jul. 23, 2021 (KR) 10-2021-0097402

(51) Int. Cl. A61N 5/06

(52) U.S. Cl. CPC A61N 5/0613; A61N 2005/0627; A61N 2005/0652; A61N 2005/0659; A61N 2005/0663

(57) ABSTRACT

A technology relating to a phototherapy device is disclosed. A therapeutic light source for outputting red light having a peak wavelength in the range of 660 nm±2% is driven by a driving pulse train having a constant pulse period during a pulse fluctuation period. The pulse period is randomly determined within a first range for each pulse fluctuation period. The optical radiant power density of a red wavelength band may be limited to be within the range of 20-100 mW/cm²-cm. A pause period of a random length in which the light source is turned off may be interposed between pulse fluctuation periods. In addition, the driving pulse train outputted during the pulse fluctuation period may have a randomly determined duty ratio.

### TECHNICAL FIELD

The present invention relates to a medical device, particularly a phototherapy device that irradiates human or animal skin with light in order to enhance metabolism or to treat diseases.

### BACKGROUND ART

In phototherapy techniques, there is a technique disclosed in the PCT publication published as WO2019-195816A1. According to the disclosed technique, the phototherapy technique may cause photons to collide with cytochrome c oxidase (CCO) molecules in mitochondria to convert adenosine monophosphate (AMP) into high-energy adenosine diphosphate (ADP), which increases cellular energy content, thereby promoting adenosine triphosphate (ATP) energy metabolism. In addition, this publication proposes red light having a wavelength of about 650 nm and near-infrared light having a wavelength of about 810 nm in a visible spectrum s suitable light for stimulating biochemical reaction through phototherapy, and provides evidence that LED light sources are suitable for this purpose. The therapeutic device disclosed in this publication uses an LED array as a radiation light source and includes a controller configured to store software defining a therapy session including on/off times for the LEDs in a memory and to load this software in order to control an LED driver according to the therapy session.

The inventors have been researching a therapeutic device similar to the above-mentioned prior art without knowledge thereof. Recent experiments in animal and human disease, particularly in the area of nerve repair as disclosed in the above publication, have confirmed the mechanism as disclosed in the publication, but during the experiments it was noted that the effect of phototherapy decreases rapidly during prolonged treatment. The decrease in effect may be a serious problem as phototherapy must be administered over a fairly long period of time for most applications. The inventors have confirmed that this decrease in effect is due to a photoreceptor, such as cytochrome c oxidase, operating according to a protective mechanism of homeostasis.

### DISCLOSURE

### Technical Problem

It is an object of the present invention to mitigate a decrease in effect in long-term phototherapy.

It is another object of the present invention to reduce the operation of a protective mechanism of homeostasis for phototherapy, thereby improving the effect of long-term phototherapy.

It is a further object of the present invention to achieve above objects through a treatment device having a simple structure.

### Technical Solution

In aspect of the present invention, a therapeutic light source may be driven by a drive pulse train having a constant pulse period during a pulse fluctuation period. The pulse period may be randomly determined within a first range for each pulse fluctuation period.

In another aspect, an optical radiant power density in a red wavelength band may be limited to within a range of 20 to 100 mW/cm²-cm.

In another aspect, a rest interval during which the light source is off may be interposed between pulse fluctuation periods. The rest interval may have a random value within a second range.

In another aspect, a driving pulse train output by a red light control unit during the pulse fluctuation period may have a duty ratio randomly determined within a third range.

In another aspect, a phototherapy device may further include a near-infrared light emitting diode configured to radiate near-infrared light having a peak wavelength of 830 nm±2%.

In another aspect, the phototherapy device may limit the duration of irradiation to the same patient in a single treatment and the number of treatments per day.

In a further aspect, the total energy of near-infrared light may be controlled so as to be 1.5 to 2.5 times higher than the total energy of red light radiated to the same patient per day.

### Advantageous Effects

Phototherapy may be a safe treatment for many diseases and may provide a variety of biological control functions, including beauty treatment, plastic surgery, metabolism improvement, and athletic ability enhancement. Furthermore, since stimulation is at the cellular/molecular level, the local therapeutic effect may spread to a systemic reaction through the bloodstream, the lymphatic system, and the nervous system.

In accordance with the present invention, these effects of phototherapy may be maintained even though phototherapy is performed over a long period of time.

### DESCRIPTION OF DRAWINGS

FIG. 1 shows the exterior of a phototherapy device according to an embodiment.

FIG. 2 is a block diagram showing the configuration of the phototherapy device according to the embodiment.

FIG. 3 is a graph schematically showing the percentage of cytochrome c oxidase activation for each wavelength band of light in an experiment.

FIG. 4 shows a part of an exemplary pulse train output by a red light control unit according to an embodiment.

FIG. 5 is a block diagram showing the configuration of an embodiment of a controller shown in FIG. 2.

FIG. 6 is a flowchart showing the configuration of a control method executed by a microprocessor in the embodiment shown in FIG. 5.

FIG. 7 is a block diagram showing the configuration of a phototherapy device according to another embodiment.

### BEST MODE

The foregoing and additional aspects are embodied in embodiments described with reference to the accompanying drawings. It is understood that components of each embodiment may be variously combined in the embodiment or may be variously combined with components of other embodiments, unless mentioned otherwise or mutually inconsistent. It should be understood that the terms or words used in the specification and appended claims should be construed based on meanings and concepts according to the technical idea of the present invention on the basis of the principle that the inventor can appropriately define the concept of terms in order to best describe their invention. Hereinafter, preferred embodiments of the present invention will be described in detail with reference to the accompanying drawings.

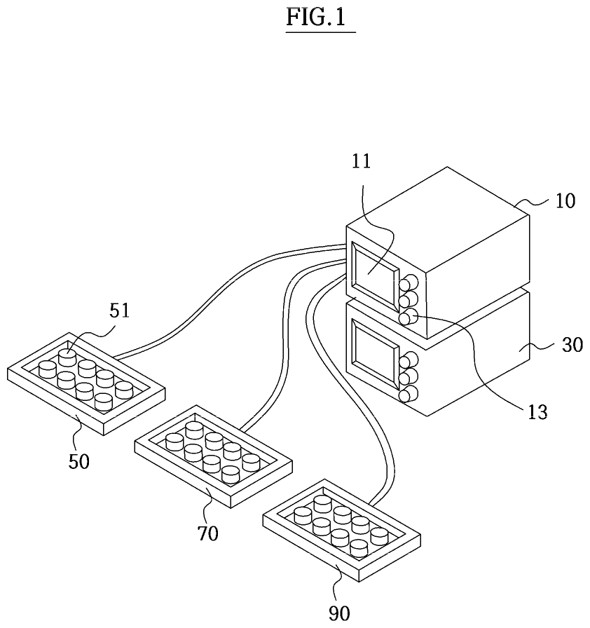

FIG. 1 shows the exterior of a phototherapy device according to an embodiment. This phototherapy device is illustrated to aid in understanding of the present invention and is not intended to limit the substantive content of the invention. As shown, the phototherapy device according to the embodiment includes a plurality of light radiation plates 50, 70, and 90 on each of which a light emitting diode array is disposed, a power supply 30, and a control device 10. The control device 10 includes a driving circuit configured to drive the light emitting diode arrays of the light radiation plates 50, 70, and 90 therein, and receives light emitting diode driving power and operating power from the power supply 30. The control device 10 includes manipulation knobs 13 and a display 11 on a front panel. An operator may use the manipulation knobs 13 to adjust driving parameters of the light emitting diode array of each of the light radiation plates 50, 70, and 90. The operator may check the driving parameters set for each of the light radiation plates 50, 70, and 90 on the display 11. The light radiation plates 50, 70, and 90 may have different output wavelengths of the light emitting diodes. For example, red light emitting diodes each having a peak wavelength of 660 nm may be disposed on the light radiation plate 50, and near-infrared light emitting diodes each having a peak wavelength of 830 nm may be disposed on the light radiation plate 70. As another example, light emitting diode groups having different wavelengths may be alternately disposed on one of the light radiation plates 50, 70, and 90.

In one aspect of the present invention, a therapeutic light source is driven by a drive pulse train having a constant pulse period during a pulse fluctuation period. The pulse period is randomly determined within a first range for each pulse fluctuation period.

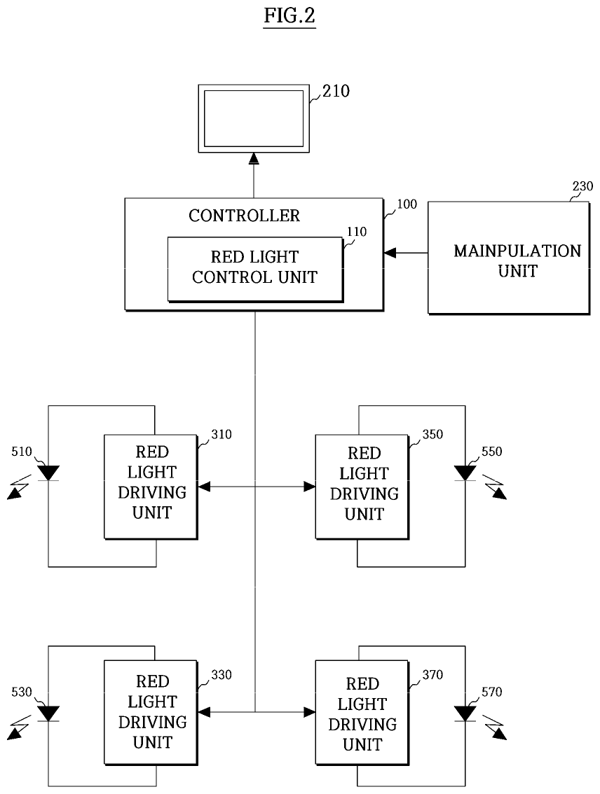

FIG. 2 block diagram showing the is a configuration of the phototherapy device according to the embodiment. As shown, the phototherapy device according to the embodiment includes a red light emitting diode 510, a red light driving unit 310, and a controller 100. In the embodiment shown, the controller 100 includes a red light control unit 110.

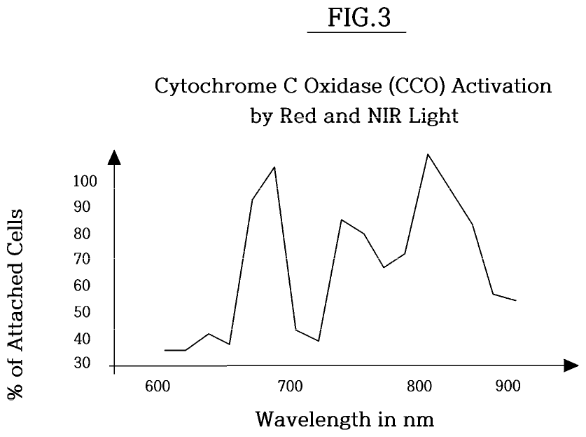

The red light emitting diode 510 outputs red light having a peak wavelength of 660 nm±2% in response to an input current. Cells in the human body have photoreceptors for specific wavelength bands of light. Examples of the photoreceptors include cytochrome C oxidase, which is found in large quantities in mitochondria, which is a small energy-producing organelle of cells, rhodopsin in eye cells, hemoglobin in blood cells, myoglobin in muscle cells, and melanin in skin cells. The photoreceptors, which absorb light to exhibit biological effects, have a specific response to low power light of specific wavelengths. FIG. 3 is a graph schematically showing the percentage of cytochrome C oxidase activation for each wavelength band of light in an experiment. Light in red and near-infrared wavelength bands penetrates deeply into the skin surface, favoring biochemical responses. There are also wavelengths of light that have negative biological effects. For example, ultraviolet light causes skin sensitization and destroys vitamin B9. Blue light is also known to have a negative effect on the brain's suprachiasmatic nucleus.

The present invention primarily targets, but is not limited to, cytochrome c oxidase in mitochondria. Mitochondria need to efficiently combine with oxygen for the production of ATP, and cytochrome c oxidase acts as a terminal oxidizing engages in oxidative phosphorylation. Red or near-infrared light stimulates this reaction, increasing the efficiency of ATP production. Another effect is a hormesis mechanism. Retrograde signaling in mitochondria caused by red or near-infrared light radiation is a type of stress, similar to exercise, which activates many cellular defense systems, enhancing anti-inflammatory and antioxidant defenses.

Mitochondria are concentrated in the heart, liver, brain, and kidney tissues, and this type of light therapy may be effective on these organs. Another study has shown that light in the red and near-infrared wavelengths may restore muscle function, improve brain function, promote recovery from traumatic injury, and relieve pain.

The red light driving unit 310 drives the red light emitting diode 510. A diode driving element is a switching element, such as a field effect transistor (FET), which switches the current supplied from the power supply in response to a control signal, such as a gate voltage.

In the embodiment shown, the controller 100 is implemented by an integrated circuit including a microprocessor, a memory, and a peripheral circuit such as a programmable counter. The functions of the controller 100 are achieved by the microprocessor reading and executing program instructions stored in the memory. In the embodiment shown, the controller 100 includes a red light control unit 110. In an aspect, the red light control unit 110 is configured to output a pulse train having a pulse period randomly determined within a first range during a pulse fluctuation period to the red light driving unit.

The pulse fluctuation period may be, for example, 1 second. The pulse period during the pulse fluctuation period is randomly determined within the first range. During the pulse fluctuation period, however, a switching control signal for the red light driving unit output by the red light control unit 110 has a constant frequency. For example, the first range may be 10 to 20 pulses per second (pps) or a period range of 0.167 to 0.333 sec.

In the same manner as in other biological stimuli, such as drugs, light stimulation, when applied continuously and repeatedly, triggers a protective mechanism of homeostasis of the body. Homeostasis is the ability or tendency of a body or a cell to maintain an appropriate level of equilibrium in response to external changes and internal demands. “Homeostasis,” Biology Online, Retrieved Oct. 27, 2019, describes homeostasis as being maintained through a regulatory mechanism consisting of three general elements: a receptor, a control center, and an effector. A homeostatic mechanism may be in the form of a positive or negative loop. Positive feedback results in more stimulus or acceleration of the process, whereas negative feedback results in inhibition of the stimulus or decrease of the process. By randomly varying the light radiation period of the red light emitting diode for each pulse fluctuation period, light radiation is perceived as new stimulus with respect to the protective mechanism of homeostasis of the body for each light radiation period, and operation of the mechanism may be reduced.

In the embodiment shown, the red light control unit 110 may include switching control program instructions configured to supply a switching control signal to the red light driving unit 310. In an embodiment, the switching control program instructions may include program instructions configured to output a pulse train to an output port at a constant frequency determined by a value set in a frequency setpoint. For example, the program instructions may generate such a pulse train using a counter circuit included in the controller 100. As another example, the program instructions may generate the pulse train by counting an internal clock of the microprocessor. This pulse train is output to the red light driving unit 310 as a switching control signal.

In addition to the red light driving unit 310 and the red light emitting diode 510, the phototherapy device according to the embodiment shown in FIG. 2 further includes three red light driving units 330, 350, and 370 and corresponding red light emitting diodes 530, 550, and 570. Depending on the application, two or three or more sets of red light driving units and red light emitting diodes may be provided. As shown in FIG. 1, these light emitting diodes may be disposed to constitute a single light radiation plate. In addition, a plurality of light radiation plates may be provided. The red light emitting diodes disposed on the single light radiation plate may flicker synchronously or may flicker asynchronously with different parameters. For the plurality of light radiation plates, the red light emitting diodes may flicker synchronously or asynchronously, and may be driven by different flickering parameters.

Additionally, the phototherapy device may include a manipulation unit 230 and a display 210. For example, the manipulation unit 230 and the display 210 may be integrated into a touch display. A user may select an operating mode necessary for driving red light or may adjust the optical radiant power density through the manipulation unit 230, and may check the operating status or setting status of the phototherapy device on the display 210.

In another aspect, the optical radiant power density in the red wavelength band is limited to within a range of 20 to 100 mW/cm²-cm. As used herein, the optical radiant power density may be an instantaneous density value or an average value over time. Photoreceptors in the body respond specifically to low power light within this range. The optical radiant power density is preferably within a range of +12 degrees, more preferably within a range of +10 degrees, of the light radiation angle of the light emitting diode with respect to the skin surface.

In the embodiment shown, the red light control unit 110 is configured to generate a driving pulse train such that the optical radiant power density of the light output by the red light emitting diode 510 is within a range of 20 to 100 mW/cm²-cm. That is, the red light control unit 110 adjusts the duty ratio of the pulse train such that the time-averaged optical radiant power density is within a range of 20 to 100 mW/cm²-cm. The optical radiant power density is highly dependent on the selection of the light emitting diode, since the radiant power density of commercially available light emitting diodes varies significantly depending upon wavelength bands.

In another aspect, a rest interval in which the light source is turned off may be inserted between the pulse fluctuation periods. The rest interval may have a random value within a second range. For example, the rest interval may be a value randomly determined within a range of 0 to 33 msec. By inserting a rest interval having a random length between pulse fluctuation periods, the time interval between outputting pulses having the same frequency may be varied. This may be more effective in inhibiting the operation of a protective mechanism of homeostasis.

In another aspect, the driving pulse train output by the red light control unit 110 during the pulse fluctuation period may have a duty ratio randomly determined within a third range. In an embodiment, the third range may include an on time of 500 to 1000 msec, an off time of 5 to 35 msec, and a duty ratio of about 90 to 95.5%.

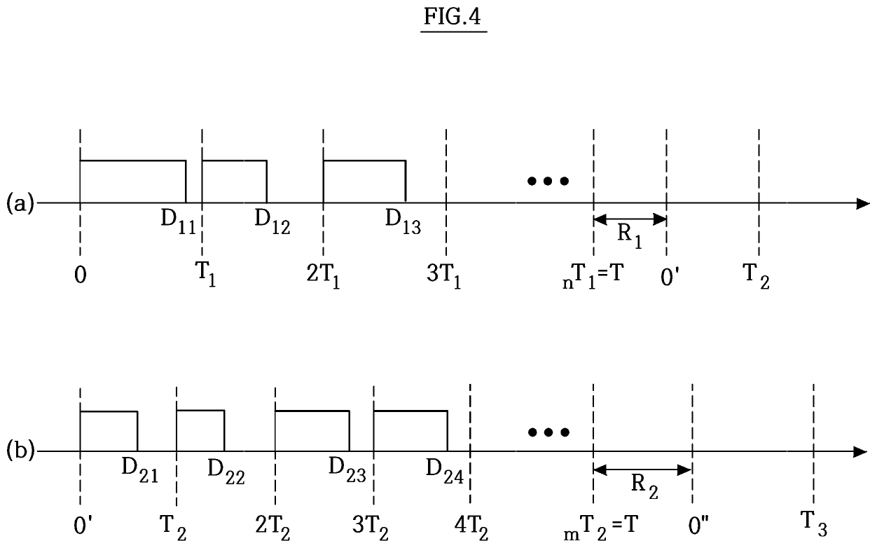

FIG. 4 shows a part of an exemplary pulse train output by a red light control unit according to an embodiment. Aspects of the present invention will be described through a pulse train waveform with reference to FIG. 4. In FIG. 4, (a) shows a pulse train during a first period, i.e., from time 0 to T, and (b) shows a pulse train during a second period, i.e., from new time 0′ to T, with a rest interval R₁ elapsed after the first period T.

In an aspect, the therapeutic light source is driven by a driving pulse train having a constant pulse period during a pulse fluctuation period. In an exemplary waveform shown, n pulse trains are output with a constant pulse period during a first period T, and m pulse trains are output with a constant pulse period during a second period T. In an aspect, the pulse period is randomly determined within a first range for each pulse fluctuation period. In an exemplary waveform shown, a first pulse fluctuation period T has a value of T₁ and a second pulse fluctuation period T has a value of T₂. Accordingly, n pulses are output during the first pulse fluctuation period T, and m pulses during the second pulse fluctuation period T.

In another aspect, a rest interval during which the light source is off may be interposed between pulse fluctuation periods. The rest interval may have a random value within a second range. In an exemplary waveform shown, a rest interval of R₁ is inserted between a first pulse fluctuation period T and a second pulse fluctuation period T, and a rest interval of R₂ is inserted between the second pulse fluctuation period T and a third pulse fluctuation period T₁. Each of R₁ and R₂ may be randomly determined within a range of 0 to 33 msec.

In another aspect, a driving pulse train output by the red light control unit 110 during a pulse fluctuation period may have a duty ratio randomly determined within a third range. In an exemplary waveform shown, pulse trains output with a period of T₁ during a first pulse fluctuation period T have randomly determined duty ratios of D₁₁, D₁₂, D₁₃, . . . , and pulse trains output with a period of T₂ during a second pulse fluctuation period T have randomly determined duty ratios of D₂₁, D₂₂, D₂₃, The values of D₁₁, D₁₂, D₁₃, . . . or D₂₁, D₂₂, D₂₃, . . . may be values randomly determined within a certain range, for example within a range of 90 to 95.5%.

In another aspect, the optical radiant power density in the red wavelength band is limited to within a range of 20 to 100 mW/cm²-cm. When the pulse period during the next pulse fluctuation period is randomly determined within the first range, therefore, a set of duty values corresponding to the number of pulses according to the determined pulse period is preferably randomly determined in advance, based on the optical radiant power density according to the specifications of the light emitting diode. The total ON time duration during the pulse fluctuation period may be determined based on the value of the optical radiant power density according to the specifications of the light emitting diode, and the determined total ON time duration may be divided by the total number of pulses in the pulse period while being randomly divided, whereby a set of duty values corresponding to the number of pulses may be predetermined.

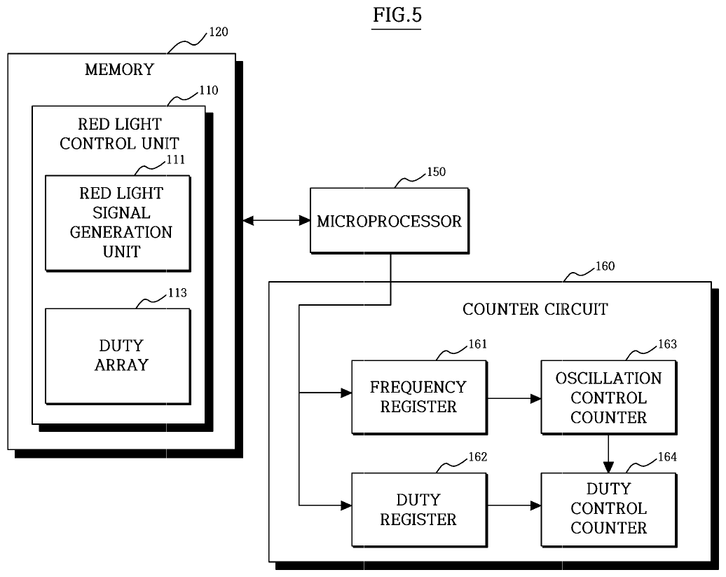

FIG. 5 is a block diagram showing the configuration of an embodiment of the controller shown in FIG. 2. In the embodiment, the controller includes a microprocessor 110, a memory 510, and a counter circuit 160. The memory 510 stores a set of program instructions constituting the red light control unit 110 in FIG. 2. The red light control unit 110 includes a red light signal generation unit 111, which is a set of program instructions, and a duty array 113, in which duty parameters of pulse trains during a single pulse fluctuation period or a plurality of pulse fluctuation periods are stored. The counter circuit 160 includes an oscillation control counter 163 and a duty control counter 164.

The oscillation control counter 163 controls the frequency or period of a pulse train during one pulse fluctuation period. A frequency register 161 stores a parameter related to the frequency value during one pulse fluctuation period, such as a count value related to the period of one pulse. The oscillation control counter 163 is controlled by the frequency register 161. For example, the oscillation control counter 163 starts counting upon instruction from the microprocessor 150 and outputs one pulse having a constant duty each time the count value related to the pulse period is reached. Accordingly, the controller 100 may generate and output a pulse train having a frequency designated by the frequency register 161 under the control of the microprocessor 150.

The duty control counter 164 implements a duty value of one pulse. A duty register 162 stores parameters related to the duty value of one pulse, such as a count value related to a pulse length and a count value related to on-time of the pulse. The duty control counter 164 is controlled by the duty register 162. In an embodiment, the duty control counter 164 is connected to start counting on a rising edge of the output pulse of the oscillation control counter 163 and to end counting on a falling edge thereof. The duty control counter 164 outputs a “1” while starting counting, transitions the output state to “0” when the count value reaches a count value related to the on-time, and transitions the state back to “1” when the count value reaches a count value related to the pulse length. Accordingly, the controller 100 may generate and output pulse trains having a frequency designated by the frequency register 161 and a duty designated by the duty register 162 under the control of the microprocessor 150.

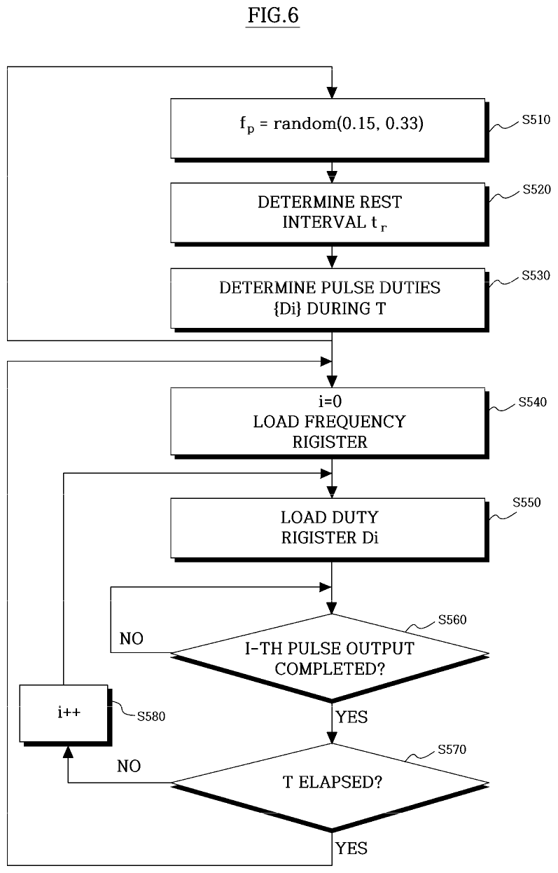

FIG. 6 is a flowchart showing the configuration of a control method executed by the microprocessor in the embodiment shown in FIG. 5. As shown, the microprocessor accesses the red light control unit 110 and executes program instructions according to the program control flow thereof.

First, pulse train parameters are calculated through steps S510, S520, and S530. First, a pulse period during one pulse fluctuation period, f_{P}, is determined and stored as a random value within a first range, for example in a range of 0.15 sec to 0.33 sec, using a random function (step S510). Subsequently, a value of tᵣ, the length of a rest interval to be inserted after the current pulse fluctuation period, is determined and stored as a random value within a second range using a random function (step S520). Subsequently, pulse duty values corresponding to the number of pulses determined according to the pulse period determined by f_{P} are randomly generated and stored within a third range (step S530). From unique radiant power density values of red light emitting diodes, the number and spacing of the red light emitting diodes used, the previously determined rest interval value tᵣ, and the pulse period, f_{P}, the total sum of the turn-on times for each pulse of the red light emitting diodes required to achieve a target optical radiant power density value determined according to the application of the phototherapy device may be calculated. Subsequently, duty ratio values {Di} for each pulse may be determined by randomly dividing the total sum time value by the total number of pulses during the pulse fluctuation period under a specific constraint, e.g., within a third range. The determined duty ratio values are stored in the duty array 113 of the memory 120. The duty array 113 may store the duty ratio values {Di} during a single pulse fluctuation period or during a plurality of pulse fluctuation periods. When generation and output of pulse trains during a given pulse fluctuation period are completed, corresponding duty array regions are either initialized or overwritten with duty ratio values {Di} during the next calculated pulse fluctuation period.

Subsequently, a loop parameter i is initialized to 0. In addition, the determined f_{P} value is loaded into the frequency register 161 (step S540). Subsequently, Di is extracted from the duty values stored in the duty array 113 of the memory 110 and loaded into the duty register. The oscillation control counter 163 and the duty control counter 164 are operated as described above, and when the output of an i-th pulse is completed, the microprocessor 150 detects the same from a trigger signal output by the duty control counter 164 at that moment, whereby the value of the loop parameter i is increased and the next Di value is extracted from the duty array 113 and loaded into the duty register. When both the generation and output of the pulse train during one pulse fluctuation period are completed, the microprocessor 150 detects the same from a trigger signal output by the oscillation control counter 163, reads the f_{P} value of the next pulse fluctuation period from the memory 120, and loads the same into the frequency register 161 (step S540). By repeating the above process, a pulse train controlling flickering of the red light emitting diode is generated and output by the controller 100.

In FIG. 5, the microprocessor 150 may wait while the counter circuit 160 generates one pulse train. In the meantime, the microprocessor 150 may repeatedly perform steps S510, S520, and S530, and may store the resulting f_{P}, tᵣ, and {Di} values in the memory 120. Accordingly, the controller 100 may generate and output a compliant pulse train without interruption.

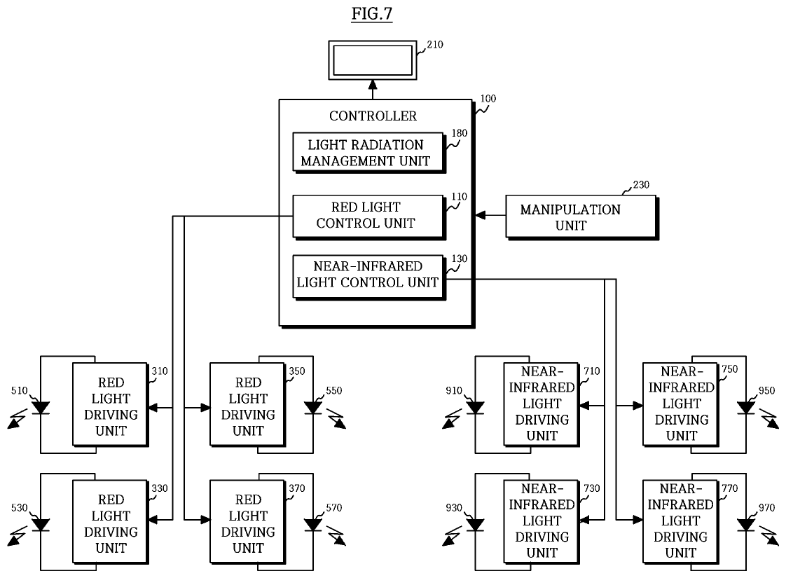

In another aspect, the phototherapy device may further include a near-infrared light emitting diode configured to radiate near-infrared light having a peak wavelength of 830 nm±2%. FIG. 7 is a block diagram showing the configuration of a phototherapy device according to another embodiment. The embodiment shown includes the configuration shown in FIG. 2, and further includes configurations for radiating near-infrared light.

In the embodiment shown, the aspects of the inventions described in claims 1 to 4 may be equally applied to the configurations of near-infrared light driving units 710, 730, 750, and 770, near-infrared light emitting diodes 910, 030, 950, and 970, and a near-infrared light control unit 130 except that light wavelengths are different. Since the near-infrared light emitting diode, which has a peak wavelength of 830 nm, has different radiant power density than the red light emitting diode, however, the range of the duty ratio of the pulse may need to be adjusted accordingly.

In another aspect, the phototherapy device may limit the total amount of energy radiated to the same patient in a single treatment. To this end, the duration of a single treatment and the number of treatments per day for the same patient may be limited. This limits the total energy supplied per day or one phototherapy treatment in order to effectively mitigate the operation of the protective mechanism of homeostasis of the body.

In another aspect, the total energy of the near-infrared light may be controlled so as to be 1.5 to 2.5 times higher than the total energy of the red light radiated to the same patient per day. Since the depth to which the near-infrared light penetrates into the body through the skin is 5 to 7 cm, which is greater than the depth to which the red light penetrates into the body through the skin, which is 2 to 5 cm, more energy must be supplied in consideration of the dispersion of energy with depth. Therefore, it is preferable to increase the optical radiant power density of the near-infrared light so as to be 1.5 to 2.5 times higher than the optical radiant power density of the red light

In order to block the operation of the protective mechanism of homeostasis, the total amount of the optical radiant energy per day is preferably limited to less than 96 J/day for the red light and less than 240 J/day for the near-infrared light in the embodiment shown.

The present invention has been described above based on the embodiments with reference to the accompanying drawings, but is not limited thereto, and should be construed to encompass a variety of variations that will be apparent to those skilled in the art. The claims are intended to cover such variations.

### INDUSTRIAL APPLICABILITY

The present invention provides a medical device, particularly a phototherapy device that irradiates human or animal skin with light in order to enhance metabolism or to treat diseases.

1. A phototherapy device configured to supply light energy to an ATP energy metabolism process in intracellular mitochondria, the phototherapy device comprising:
a red light emitting diode configured to output red light having a peak wavelength of 660 nm±2%; and
a red light driving unit configured to drive the red light emitting diode; and
a controller comprising a red light control unit configured to output a driving pulse train having a pulse period randomly determined within a first range during a pulse fluctuation period to the red light driving unit.

2. The phototherapy device according to claim 1, wherein the red light control unit is configured to generate the driving pulse train such that an optical radiant power density of the light output by the red light emitting diode is within a range of 20 to 100 mW/cm²-cm.

3. The phototherapy device according to claim 1, wherein the red light control unit is configured to output an off signal during a rest interval having a random value within a second range as a period between the pulse fluctuation period and a next pulse fluctuation period.

4. The phototherapy device according to claim 1, wherein the red light control unit is configured such that the driving pulse train output during the pulse fluctuation period has a duty ratio randomly determined within a third range.

5. The phototherapy device according to claim 1, further comprising:
a near-infrared light emitting diode configured to radiate near-infrared light having a peak wavelength of 830 nm±2%; and
a near-infrared light driving unit configured to drive the near-infrared light emitting diode, wherein
the controller further comprises a near-infrared light control unit configured to output a driving pulse train having a pulse period randomly determined within a third range during the pulse fluctuation period.

6. The phototherapy device according to claim 5, wherein the near-infrared light control unit is configured to output an off signal during a rest interval having a random value within a fourth range as a period between the pulse fluctuation period and a next pulse fluctuation period.

7. The phototherapy device according to claim 5, wherein the near-infrared light control unit is configured such that the driving pulse train output during the pulse period has a duty ratio randomly determined within a fifth range.

8. The phototherapy device according to claim 5, wherein the light near-infrared control unit is configured to generate the driving pulse train such that an optical radiant power density of the light output by the near-infrared light emitting diode is within a range of 20 to 100 mW/cm²-cm.

9. The phototherapy device according to claim 5, further comprising a light radiation management unit configured to control the red light control unit and the near-infrared light control unit in order to limit a duration of irradiation to the same patient in a single treatment and the number of treatments per day.

10. The phototherapy device according to claim 9, wherein the light radiation management unit controls the red light control unit and the near-infrared light control unit such that the total energy of the near-infrared light is 1.5 to 2.5 times higher than the total energy of the red light radiated to the same patient per day.

11. A method of controlling a phototherapy device comprising: a red light emitting diode configured to output red light having a peak wavelength of 660 nm±2%; and a red light driving unit configured to drive the red light emitting diode, the phototherapy device being configured to supply light energy to an ATP energy metabolism process in intracellular mitochondria, wherein the method outputs a driving pulse train having a pulse period randomly determined within a first range during a pulse fluctuation period to the red light driving unit.

12. The method according to claim 11, wherein the driving pulse train is configured such that an optical radiant power density of the light output by the red light emitting diode is within a range of 20 to 100 mW/cm²-cm.

13. The method according to claim 11, wherein the method outputs an off signal during a rest interval having a random value within a second range as a period between the pulse fluctuation period and a next pulse fluctuation period.

14. The method according to claim 11, wherein the driving pulse train output during the pulse fluctuation period has a duty ratio randomly determined within a third range.

15. The method according to claim 11, wherein
the phototherapy device further comprises a near-infrared light emitting diode configured to radiate near-infrared light having a peak wavelength of 830 nm±2%, and
the method outputs a driving pulse train having a pulse period randomly determined within a third range during the pulse fluctuation period to the near-infrared light emitting diode.

16. The method according to claim 15, wherein the method outputs an off signal during a rest interval having a random value within a fourth range as a period between the pulse fluctuation period of the driving pulse train output to the near-infrared light emitting diode and a next pulse fluctuation period.

17. The method according to claim 15, wherein the driving pulse train output to the near-infrared light emitting diode during the pulse fluctuation period is configured to have a duty ratio randomly determined within a fifth range.

18. The method according to claim 15, wherein the driving pulse train is configured such that an optical radiant power density of the light output by the near-infrared light emitting diode is within a range of 20 to 100 mW/cm²-cm.

19. The method according to claim 15, wherein the method limits a duration of irradiation to the same patient in a single treatment and the number of treatments per day.

20. The method according to claim 19, wherein the method performs control such that the total energy of the near-infrared light is 1.5 to 2.5 times higher than the total energy of the red light radiated to the same patient per day.
